(19) United States
(12) Patent Application Publication
(10) Pub. No.: US 2016/0181648 A1
(43) Pub. Date: Jun. 23, 2016

(54) FUEL BIOCELL

(71) Applicant: COMMISSARIAT A L'ENERGIE ATOMIQUE ET AUX ENERGIES ALTERNATIVES, Paris (FR)

(72) Inventors: Aurélien AUGER, Grenoble (FR); Raoudha HADDAD, Grenoble (FR)

(73) Assignee: COMMISSARIAT ÀL'ENERGIE ATOMIQUE ET AUX ENERGIES ALTERNATIVES, Paris (FR)

(21) Appl. No.: 14/909,214

(22) PCT Filed: Jul. 30, 2014
(86) PCT No.: PCT/IB2014/063553

§ 371 Date: Feb. 1, 2016

(30) Foreign Application Priority Data
Jul. 31, 2013 (FR) 13 57566

(51) Int. Cl. H01M 8/16; H01M 4/86; H01M 4/90; C12N 11/08; H01M 4/88

(52) U.S. Cl. CPC H01M 8/16; C12N 11/08; H01M 4/8828; H01M 4/9075; H01M 4/9008; H01M 4/8663; H01M 2250/00

(57) ABSTRACT

The invention relates to nanoparticles, preferably metal oxides, at least partially coated on the surface by a silylated polymer, and functionalised by at least one molecule of an oxidation-reduction mediating agent, characterised in that the immobilisation of said molecule(s) of said mediating agent on the surface of said nanoparticles is enabled by non-covalent bonds, preferable π-π type interactions, established between ethylenic, acetylenic and/or aromatic patterns, respectively present in the region of said silylated polymer and said molecule(s) of the oxidation-reduction mediating agent.

The present invention relates to the field of fuel cells. More particularly, the present invention relates to nanoparticles functionalized at the surface with at least one redox mediator, to the process for preparing them and to their use for preparing an electrode, especially for a fuel cell, and also to the preparation of a biocell, especially a glucose biocell.

A fuel biocell, or enzymatic biocell, is a cell in which at least one of the two catalysts is enzymatic. The advantage of using enzymatic catalysts lies in their ability to oxidize or reduce organic molecules specifically. The second advantage of enzymatic catalysts is that of exploiting their catalytic performance in physiological electrolytic media.

As examples of fuel biocells, mention may be made of the biocells described in WO 2011/117357 and WO 2005/096430.

Many problems remain to be solved in the production of enzymatic biocells to allow the development of these energy sources. In particular, it is necessary to increase the power of the current biocells. Various routes have been attempted in this respect, such as improving the enzyme immobilization techniques, modifying the chemical structure of enzymes, or improving the electron transfer between the active center of the enzyme and the surface of the electrode.

Numerous studies have been conducted in recent years on the development of novel energy conversion systems. Although several fuels, such as methanol, ethanol and hydrogen, have been extensively studied, glucose remains the ideal fuel for medical applications. The glucose/oxygen fuel cell, or biocell, constitutes a very promising route for providing an energy supply to devices that may be implanted into a living being, in particular medical devices, without an external fuel feed being necessary. Glucose is a constituent of physiological fluid in mammals (5 mM in human blood), and it is nontoxic. The development of such a cell technology in which the fuel is glucose and the oxidant is molecular oxygen would allow a major advance in the development of implantable devices, especially in the medical field, such as those devoted to detecting diabetes, or for supplying pacemakers or auditive or ocular devices.

Glucose oxidase (GOx), a homodimeric enzyme, is undoubtedly the enzyme most widely used in the design of glucose/oxygen biocells, especially with the keen interest observed in the development of glucose metabolic biosensors. Each of these subunits comprises a redox cofactor responsible for its enzymatic activity toward D-glucose: flavine adenine dinucleotide (FAD). GOx has very high selectivity toward glucose. Specifically, it preferentially oxidizes only D-glucose. Furthermore, it has noteworthy activity and stability under physiological temperature and pH conditions (37° C., ˜7.4). These two characteristics make this protein a favored candidate for the development of implantable glucose biocells.

As indicated previously, the development of a biocell involves being able efficiently to transfer electrons from the catalytic site of the enzyme under consideration to the electrode of the biocell. As regards GOx, its FAD redox center is deeply buried in the protein cage of the enzyme. Although this localization gives the enzyme good stability, it creates difficulty regarding exploitation of direct electron transfer from FAD to the electrode. Thus, under normal conditions, the transfer of electrons between the active site of GOx and an electrode is a very slow or even nonexistent process. This phenomenon is due to the very great distance between the active site of the enzyme and the electrode. However, electron transfer between the active site and the electrode may be facilitated if an electron acceptor is used as mediator.

The problem of improving the electron transfer between the active center of an enzyme and a surface of the electrode is also encountered in the field of biosensors and biodetectors.

Wilson et al. (Biosensors & Bioelectronics, 1992, 7:165) review biosensors using glucose oxidase (GOX).

Teng et al. (Biosensors & Bioelectronics, 2011, 26:4661) describe ZnO nanotubes that are surface-modified with ferrocene. The ZnO nanotubes are surface-modified with tetraethyl orthosilicate (TEOS) in the presence of aqueous ammonia. Onto these modified nanotubes is covalently grafted a ferrocene monocarboxylic acid. The nanotubes are also coupled with an antibody and are used in an electrochemical immunoassay process for antigen detection.

Qiu et al. (Biosensors & Bioelectronics, 2009, 24:2920) describe a chitosan/ferrocene/Au nanoparticles/glucose oxidase composite film, in which the ferrocene is covalently bonded to the chitosan, which is then adsorbed onto the gold nanoparticles. The film is deposited on an electrode for the detection of glucose.

Liang et al. (Electrochimica Acta, 2012, 69:167) describe Fe₂O₃ nanoparticles that are covalently surface-modified with ferrocene by means of a click chemistry method resulting from the reaction between the thiol functions introduced at the surface of the nanoparticles and a vinyl group borne by the ferrocene. The nanoparticles obtained are used on an electrode in the presence of glucose oxidase for the detection of glucose.

Qiu et al. (Biosensors & Bioelectronics, 2009, 24:2649) describe Fe₂O₃ nanoparticles covered with gold (Fe₂O₃@Au), covalently surface-modified with ferrocene (Fc), obtained by reacting Fe₂O₃@Au nanoparticles with HS(CH₂)₆Fc. The nanoparticles obtained are used on an electrode for the detection of dopamine.

Hu et al. (J. Mol. Catal. B-Enzyme., 2011, 72:298) describe an electrode comprising at its surface, deposited by adsorption or by electrodeposition, especially multiwalled carbon nanotubes, ZnO and GOx nanoparticles. The electrode is used for detecting glucose.

However, there is still a need to improve the electron transfer between an enzyme that is capable of catalyzing an oxidation reaction, and in particular of oxidizing glucose, and an electrode so as to obtain a satisfactory yield for the development of a fuel biocell, and especially a glucose biocell.

There is also a need for modified nanoparticles, which may be used for the preparation of an electrode of a fuel biocell, and which improve the electron transfer from the active center of an enzyme to the electrode.

There is also a need for nanoparticles functionalized with a redox mediator.

There is also a need for a simple and efficient process for preparing such nanoparticles.

There is also a need for a simple, efficient process for preparing in good yield nanoparticles functionalized with a redox mediator.

There is also a need for electrodes formed especially by means of an enzyme that is capable of catalyzing an oxidation reaction, and in particular of oxidizing glucose, in which the electron transfer between the catalytic site of the enzyme and the electrode is improved.

There is also a need for modified nanoparticles, which may be used for the preparation of an electrode of a fuel biocell, which are stable over time, which can be stored in a dry form and can be rapidly and simply dissolved for the preparation of the electrode.

There is also a need for fuel biocells whose energy yield is improved.

There is also a need for fuel biocells whose various components can be easily recycled, in particular the various components constituting an enzymatic catalyst electrode.

The object of the present invention is to satisfy these needs.

Thus, according to one of its first subjects, the present invention relates to nanoparticles, preferably of metal oxides, at least partially surface-coated with a silylated polymer, and functionalized with one or more molecules of at least one redox mediator, characterized in that the immobilization of said molecule(s) of said mediator at the surface of said nanoparticles takes place via non-covalent bonds, preferably interactions of π-π type, established between ethylenic, acetylenic and/or aromatic units, respectively, present on said silylated polymer and on said redox mediator molecule(s).

Unexpectedly, and as detailed in the examples present below, the inventors have observed that such nanoparticles, when they are used on an electrode, in the presence of an enzyme that is capable of performing an oxidation reaction, and in particular capable of oxidizing glucose, and more particularly a glucose oxidase (GOx), promote the transfer of electrons to the electrode.

As also shown in the examples below, the use of an electrode comprising these nanoparticles, preferably adsorbed onto multiwalled carbon nanotubes in the presence of an enzyme that is capable of catalyzing an oxidation reaction, such as a GOx, in a fuel biocell makes it possible to substantially improve the energy yield of this biocell.

Finally, as shown in the examples detailed below, the nanoparticles, the electrodes and the biocells comprising them especially prove to be simple to produce, stable over time, and can be readily recycled.

According to another of its subjects, the present invention relates to agglomerates of nanoparticles of the invention, and preferably comprising at the surface at least one water-soluble polymer, as described below.

According to another of its subjects, the present invention relates to a support material on the surface of which is adsorbed at least one nanoparticle of the invention.

According to another of its subjects, the present invention relates to an ink comprising at least nanoparticles of the invention or a support material of the invention.

According to another of its subjects, the present invention relates to an electrode totally or partially formed from nanoparticles of the invention or from a support material of the invention.

According to another of its subjects, the present invention relates to a fuel biocell comprising, as anode, nanoparticles of the invention, a support material of the invention, an ink of the invention, said ink being dried, or at least one electrode of the invention.

According to another of its subjects, the present invention relates to a process for preparing nanoparticles of the invention, comprising at least the steps consisting in:

a—providing nanoparticles, preferably of metal oxide, dispersed in an organic solvent,

b—placing said nanoparticles in contact with at least one silylated polymer precursor comprising at least one ethylenic, acetylenic and/or aromatic unit, under conditions favorable for the formation of a silylated polymer bearing reactive ethylenic, acetylenic and/or aromatic unit(s), and for its deposition at least partially at the surface of said nanoparticles,

c—placing the nanoparticles obtained in step b— in contact with at least one redox mediator molecule bearing at least one ethylenic, acetylenic and/or aromatic unit, under conditions suitable for immobilizing molecule(s) of said mediator at the surface of said nanoparticles via the establishment of non-covalent bonds, preferably interactions of π-π type, established between the ethylenic, acetylenic and/or aromatic units respectively present on said silylated polymer and on the redox mediator molecule(s).

For the purposes of the invention, the term “reagent(s)” with regard to the ethylenic, acetylenic and/or aromatic unit(s) of a silylated polymer at the surface of a nanoparticle is intended to qualify one or more ethylenic, acetylenic and/or aromatic unit(s) arranged so as to allow them to form non-covalent bonds, preferably interactions of π-π type, with one or more ethylenic, acetylenic and/or aromatic units present on a redox mediator used to functionalize a nanoparticle.

According to another of its subjects, the present invention relates to a process for preparing an agglomerate of the invention, comprising at least the steps consisting in:
providing a dispersion of nanoparticles of the invention, and preferably comprising at the surface at least one water-soluble polymer, as described below,
performing the dropwise dipping of said dispersion, in liquid nitrogen, to obtain a solid material,
recovering by filtration the solid material thus obtained, and, where appropriate
lyophilizing the solid material thus recovered.

According to another of its subjects, the present invention relates to a process for preparing an electrode of the invention, comprising at least one step that consists in depositing on to a support, in particular a glass-like carbon support or an anionic and/or cationic membrane, preferably a membrane of a fluoropolymer based on sulfonated tetrafluoroethylene copolymer, an ink of the invention.

The present invention advantageously makes it possible to prepare, simply and reproducibly, nanoparticles that may be used for the preparation of an electrode of a fuel biocell, and improving the electron transfer from the active center of an enzyme to the electrode.

The present invention advantageously makes it possible to provide nanoparticles that are surface-functionalized with at least one redox mediator configured to allow electron transfer between the catalytic site of an enzyme and an electrode.

The present invention advantageously makes it possible to prepare fuel biocells that have an improved energy yield, which may be used for supplying energy to devices, especially medical devices, in particular devices with a therapeutic or diagnostic function, intended to be implanted into an animal, especially a mammal, in particular a human being.

### KEYS TO THE FIGURES

FIG. 1: illustrates the interactions of a functionalized ZnO nanoparticle according to the invention with a glucose oxidase (GOx) enzyme.

FIG. 2: illustrates the structure of GOx and the principle of glucose oxidation.

FIGS. 3A, 3B and 3C: represent (3A) unmodified ZnO nanoparticles, (3B) dried and ground ZnO nanoparticles surface-modified with a silylated polymer, and (3C) ZnO nanoparticles surface-modified with a silylated polymer and functionalized with redox mediator molecules. The bar represents 30 nm. The observations are made by transmission electron microscopy (TEM).

FIG. 4: represents the operating scheme for the surface modification of a nanoparticle of the invention, ZnO nanoparticle, with a silylated polymer and its functionalization by non-covalent bonding with redox mediator molecules (ferrocene, Fc) (ZnO-Fc nanoparticle), followed by lyophilization of these nanoparticles in the presence of a water-soluble polymer (polyvinyl alcohol, PVA).

FIG. 5: illustrates the operating scheme for a glucose/oxygen biocell of the invention using a glucose oxidase at the anode.

FIGS. 6A and 6B: illustrate cyclic voltammetry curves of a glass-like carbon electrode functionalized (A) with ZnO-Fc nanoparticles alone and (B) with ZnO-Fc nanoparticles alone (curve (a)) or ZnO-Fc nanoparticles adsorbed onto multiwall carbon nanotubes (MWCNT) (MWCNT/ZnO-Fc) (curve (b)) in a phosphate buffer (100 mM) at pH 7. Scanning speed: 50 mV/s.

FIGS. 7A and 7B: illustrate (A) a linear-voltage polarization curve at 2 mV/s and (B) a corresponding power curve of a biofuel cell whose anode comprises (a) ZnO-Fc nanoparticles adsorbed onto carbon nanotubes in the presence of GOx (CNTs/ZnO-Fc/GOx) or (b) carbon nanotubes in the presence of ferrocene and GOx (CNTs/Fc/GOx), and whose cathode is based on platinum (C—Pt).

FIG. 8: illustrates a discharge curve of a biofuel cell of the invention, whose anode comprises ZnO-Fc nanoparticles adsorbed onto carbon nanotubes in the presence of GOx (CNTs/ZnO-Fc/GOx) subjected to a voltage of 200 mV.

FIG. 8: illustrates the possible interactions of functionalized ZnO nanoparticles according to the invention with GOx molecules.

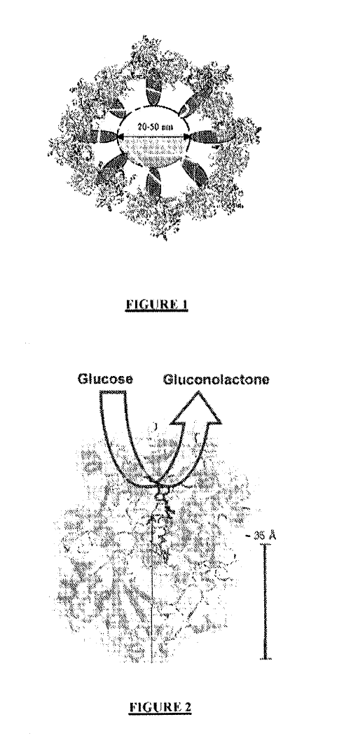

### NANOPARTICLES

### Nanoparticles

The nanoparticles of the invention are at least partially coated, or modified, at the surface, with a silylated polymer, and functionalized with one or more molecules of at least one redox mediator. Immobilization of the mediator molecule(s) at the surface of the nanoparticles takes place via non-covalent bonds, established between ethylenic, acetylenic and/or aromatic units, which are present, respectively, on the silylated polymer and on the redox mediator molecule(s).

As pointed out above, immobilization of the mediator molecule(s) at the surface of the nanoparticles advantageously takes place via interactions of π-π type.

According to one embodiment, the nanoparticles of the invention may be metal oxides.

According to one embodiment, the nanoparticles that are suitable for use in the invention may be semiconductive nanoparticles.

Preferably, the nanoparticles that are suitable for use in the invention may be nanoparticles chosen from SiO₂, MgO, Al₂O₃, ZnO, CeO₂, TiO₂, and ZrO₂ nanoparticles, and mixtures thereof. More preferably, the nanoparticles of the invention may be ZnO nanoparticles.

The nanoparticles that are suitable for use in the invention may have a mean size diameter ranging from 2 to 50 nm, preferably ranging from 10 to 50 nm, preferably ranging from 15 to 40 nm and even more preferentially ranging from 20 to 30 nm.

The mean size diameter of the nanoparticles of the invention may be measured via any method known to those skilled in the art. In particular, the mean size diameter of the nanoparticles may be determined by photon correlation spectroscopy (PCS), or may be measured directly on photographs taken by transmission electron microscopy (TEM). Such methods are known to those skilled in the art and do not need to be presented in further detail in the present text.

The nanoparticles that are suitable for use in the invention may be obtained via any technique known to those skilled in the art, and especially as illustrated in the examples of the invention.

By way of example, the ZnO nanoparticles that are suitable for use in the invention may be obtained by coprecipitation, using hydrated zinc nitrate and sodium carbonate, both in aqueous solution. The zinc nitrate solution is added dropwise to the sodium carbonate solution with stirring at room temperature. The solution obtained may then be stirred, preferably for at least 2 hours at room temperature. The precipitate obtained may be filtered off. The powder thus recovered may be dried, for example in an oven, especially at 100° C. The solid is then calcined. The calcination may be performed in air, at 400° C., for 4 hours.

### Silicon Alkoxide

The nanoparticles of the invention are at least partially coated, or modified, at the surface with a silylated polymer.

According to one embodiment of the invention, the silylated polymer may form a monomolecular layer at the surface of the nanoparticles.

The silylated polymer at the surface of the nanoparticles may be obtained by polymerization of at least one silylated polymer precursor comprising at least one ethylenic, acetylenic and/or aromatic unit. Polymerization of this silylated polymer precursor may be performed such that the silylated polymer forms a monomolecular layer at the surface of the nanoparticles.

The presence of a monomolecular layer of silylated polymer at the surface of the nanoparticles may be validated, for example, by determination of the zeta potential.

According to one embodiment, the silylated polymer precursor may be a silicon alkylalkoxide of general formula (I):

R¹R²R³SiR⁴

in which:
R¹, R² and R³, which may be identical or different, may represent a group OR⁵ with R⁵ possibly representing a saturated, linear or branched C₁ to C₄ alkyl group; or a halogen, in particular chosen from Cl and F, and
R⁴ may represent a linear, branched or cyclic C₂ to C₁₀ hydrocarbon-based group, comprising at least one ethylenic, acetylenic and/or aromatic unit, and, where appropriate, interrupted with one or more heteroatoms, such as an oxygen or a nitrogen, and preferably an oxygen, and/or bearing an oxo function.

According to a preferred embodiment, R¹, R² and R³, which may be identical or different, and preferably identical, may represent a group OR⁵, with R⁵ being as defined above or below; or a halogen chosen from Cl and F.

Preferably, R¹, R² and R³, which may be identical or different, and preferably identical, may represent a halogen chosen from Cl and F.

More preferably, R¹, R² and R³, which may be identical or different, and preferably identical, may represent a group OR⁵, with R⁵ being as defined above or below.

According to a preferred embodiment, R⁵ may represent an alkyl group chosen from a methyl, an ethyl and a propyl, and preferably chosen from a methyl and an ethyl. Preferably, R¹, R² and R³ are identical, and may be chosen from OR⁵, with R⁵ being a methyl or an ethyl.

According to one embodiment, R⁴ may be a C₂ to C₁₀ hydrocarbon-based group comprising a radical chosen from an aryl radical, in particular a phenyl or naphthyl radical, a vinyl (or ethenyl) radical, or a propenyl or isopropenyl radical, and, where appropriate, being interrupted with one or more heteroatoms, such as an oxygen or a nitrogen, and preferably an oxygen, and/or bearing an oxo function.

According to one embodiment, R⁴ may be a cyclic C₆ to C₁₀ hydrocarbon-based group comprising an aryl radical chosen from a phenyl or naphthyl radical.

According to one embodiment, R⁴ may be a linear or branched C₂ to C₈ hydrocarbon-based group comprising at least one ethylenic or acetylenic unsaturation, and being optionally interrupted with an oxygen atom and/or bearing an oxo function.

Preferably, R⁴ may be optionally interrupted with an oxygen atom and/or may bear an oxo function. More preferably, the oxygen interrupting the hydrocarbon-based chain and the oxo group may be conjugated.

According to a preferred embodiment, R⁴ may be chosen from a vinyl radical, a propenyl or isopropenyl radical, and a branched C₇ hydrocarbon-based radical, comprising at least one ethylenic unsaturation, the chain being interrupted with an oxygen atom and bearing an oxo function, the oxygen atom and the oxo function being conjugated.

According to a preferred embodiment, R⁴ may be chosen from a phenyl group, a vinyl radical, a propenyl radical, an isopropenyl radical and a radical (CH₂)₃OC(O)C(CH₃)═CH₂.

More preferably, R⁴ may be chosen from a phenyl radical, a vinyl radical, a propenyl radical and a radical —(CH₂)₃OC(O)C(CH₃)═CH₂.

More preferably, R⁴ may be chosen from a phenyl radical, a vinyl radical and a radical —(CH₂)₃OC(O)C(CH₃)═CH₂.

A silicon alkoxide that is suitable for use in the invention may be chosen from trimethoxyvinylsilane (CAS No. 2768-02-7), triethoxyvinylsilane (CAS No. 78-08-0), triethoxyphenylsilane (CAS No. 780-69-8), trimethoxyphenylsilane (CAS No. 2996-92-1), and the ester of 2-methylpropen-2-oyl and of 3-(trimethoxysilyl)propyl (CAS No. 2530-85-0), or even a mixture thereof.

Preferably, a silicon alkoxide that is suitable for use in the invention may be chosen from trimethoxyvinylsilane, triethoxyvinylsilane, triethoxyphenylsilane and trimethoxyphenylsilane, or even a mixture thereof.

Preferably, a silicon alkoxide that is suitable for use in the invention is trimethoxyvinylsilane.

### Redox Mediator Molecules

The nanoparticles that are at least partially coated, or modified, at the surface with a silylated polymer are functionalized with one or more molecules of at least one redox mediator.

A redox mediator molecule that is suitable for use in the invention may be chosen from ferrocenes; methylene green; Nile blue; macrocyclic organometallic complexes, such as porphyrins and phthalocyanins; quinones; phenoxazines, such as methylene blue or toluidine blue; tetrathiafulvalene; tetracyanoquinodimethane; benzylviologen; tris(2,2′-bipyridine)cobalt(III) perchloride; indophenols, such as dichlorophenolindophenol; and is preferably chosen from ferrocenes.

Preferably, a redox mediator molecule that is suitable for use in the invention is a ferrocene molecule.

A redox mediator molecule that is suitable for use in the invention is present in a form functionalized with at least one radical comprising at least one ethylenic, acetylenic and/or aromatic unit.

A radical comprising at least one ethylenic, acetylenic and/or aromatic unit that is suitable for use in the invention may be a C₂ to C₁₀ hydrocarbon-based group comprising a radical chosen from an aryl radical, in particular a phenyl or naphthyl radical, a vinyl radical, or a propenyl or isopropenyl radical, and, where appropriate, interrupted with one or more heteroatoms, such as an oxygen or a nitrogen, and preferably an oxygen, and/or bearing an oxo function.

In particular, such a radical may satisfy the definitions of the radical R⁴ given previously.

A radical comprising at least one ethylenic, acetylenic and/or aromatic unit may be linked directly or via a spacer to the redox mediator molecule.

A spacer that is suitable for use in the invention may be a C₁-C₁₀ carbon-based chain, optionally substituted with alkyl units, in particular of C₁ to C₄, and/or halogen units, chosen in particular from Cl and F, and/or optionally interrupted with heteroatoms, especially O or N, and/or amide, ester or ether functions. The choice of the presence and nature of a spacer arm falls within the general knowledge of a person skilled in the art. In particular, the choice of the nature of the spacer between a redox mediator molecule and a radical comprising at least one ethylenic, acetylenic and/or aromatic unit is made on the basis of the general knowledge of a person skilled in the art, taking into consideration that this spacer should not affect, at least not substantially, the ability of the ethylenic, acetylenic and/or aromatic unit(s) to bond non-covalently, and especially via interactions of π-π type, to the ethylenic, acetylenic and/or aromatic unit(s) present on the silylated polymer.

It falls within the general knowledge of a person skilled in the art, depending on the nature of the redox mediator molecule and the nature of the radical comprising at least one ethylenic and/or aromatic unit to be grafted onto the latter, to use appropriate operating conditions. These conditions therefore do not need to be presented in further detail herein.

Redox mediator molecules thus modified that are suitable for use in the invention are commercially available. As redox mediators thus modified that are suitable for use in the invention, mention may be made, in a nonlimiting manner, of vinyl ferrocene (CAS No. 1271-51-8) and ferrocenylmethyl methacrylate (CAS No. 31566-61-7).

### Water-Soluble Polymer

The nanoparticles of the invention may also comprise at the surface at least one water-soluble polymer. Such a polymer may be adsorbed at the surface of the nanoparticles. The use of a water-soluble polymer advantageously makes it possible to obtain a homogeneous dispersion in a liquid of the redox mediator molecules located at the surface of the nanoparticles of the invention. In particular, such a polymer may make it possible to promote the formation of mediators that are insoluble and indispersible.

A water-soluble polymer that is suitable for use in the invention may be chosen from polyvinyl alcohol, polyethylene-40 stearate, poly(vinylidene chloride-co-vinyl chloride), poly(styrene-co-maleic anhydride), polyvinylpyrrolidone, poly(vinyl butyral-co-vinyl alcohol-co-vinyl acetate), poly(maleic anhydride-alt-1-octadecene), poly(vinyl chloride), PEOX (poly(2-ethyl-2-oxazoline)), PLGA (poly(lactic acid-co-glycolic acid)), and mixtures thereof.

Preferably, a water-soluble polymer that is suitable for use in the invention is polyvinyl alcohol (PVA).

### Process for Preparing the Nanoparticles

A process for preparing nanoparticles of the invention may comprise at least the steps consisting in:

a—providing nanoparticles, preferably of metal oxide, dispersed in an organic solvent,

b—placing said nanoparticles in contact with at least one silylated polymer precursor comprising at least one ethylenic, acetylenic and/or aromatic unit, under conditions favorable for the formation of a silylated polymer bearing reactive ethylenic, acetylenic and/or aromatic unit(s) (i.e. capable of reacting with the redox mediator), and for its deposition at least partially at the surface of said nanoparticles,

c—placing the nanoparticles obtained in step b— in contact with at least one redox mediator molecule bearing at least one ethylenic, acetylenic and/or aromatic unit, under conditions suitable for immobilizing molecules of said mediator at the surface of said nanoparticles via the establishment of non-covalent bonds, preferably interactions of π-π type, established between the ethylenic, acetylenic and/or aromatic units respectively present on said silylated polymer and said redox mediator molecule(s).

The nanoparticles to be used in a process of the invention may especially be as defined previously.

In step a— of a process of the invention, the nanoparticles may be dispersed in an organic solvent, in a concentration ranging from 0.02 to 20 g/L, preferably ranging from 0.1 to 15 g/L, more preferably ranging from 0.5 to 10 g/L, more preferably ranging from 1 to 5 g/L and even more preferentially at a concentration of about 2 g/L.

An organic solvent that is suitable for use in the invention may be chosen especially from isopropanol, tetrahydrofuran (THF), butanol and cyclohexanol.

Preferably, an organic solvent that is suitable for use in the invention is isopropanol.

Step b— of a process of the invention advantageously makes it possible to obtain nanoparticles that are surface-modified using a silylated polymer, preferably as a monomolecular layer.

The surface modification of the nanoparticles, in step b— of a process of the invention, may be performed using a silicon alkoxide, especially as defined previously.

Such a silicon alkoxide may be used in a reaction solvent in a proportion of from 0.01 to 10 g/L, preferably from 0.05 to 8 g/L, preferably from 0.1 to 6 g/L, preferably from 0.2 to 4 g/L, more preferably from 0.4 to 2 g/L, or even from 0.6 to 1 g/L, and even more advantageously in a proportion of 0.6 g/L.

A reaction solvent that is suitable for use in the invention may be a solvent, or organic phase, which may be chosen especially from isopropanol, tetrahydrofuran (THF), butanol and cyclohexanol.

Preferably, a reaction solvent that is suitable for use in the invention is isopropanol.

According to one embodiment, in a process of the invention, step b— may be performed in the presence of a hydrolyzing agent. Such an agent may be chosen especially from aqueous ammonia, sodium hydroxide and potassium hydroxide.

Preferably, a hydrolyzing agent that is suitable for use in the invention is aqueous ammonia.

A hydrolyzing agent may be used in a proportion of from 5% to 50%, or even from 6% to 40%, or even from 7% to 30%, or even from 8% to 20%, or even advantageously in a proportion of about 10% by weight relative to the weight of the solvent.

Step b— of a process of the invention may be performed at room temperature, ranging from about 20 to 24° C., and may last from about 20 to 30 hours, and preferably about 24 hours.

Preferably, this step may be performed with stirring, according to the means usually used by a person skilled in the art in the field.

Step c— of a process of the invention advantageously makes it possible to obtain nanoparticles that are surface-functionalized with one or more molecules of a redox mediator.

This step c— may be performed using redox mediator molecules, especially as defined previously.

In step c— of a process of the invention, a redox mediator may be used in a reaction solvent in a proportion of from 0.25% to 25% by weight, relative to the weight of the solvent, or even from 0.5% to 20%, or even from 1% to 15%, or even from 2% to 10%, or even from 3% to 8%, or even from 4% to 6%, and even more advantageously in a proportion of 5% by weight, relative to the weight of the solvent.

For the implementation of a step c— of a process of the invention, the nanoparticles derived from step b— may be recovered, isolated and dispersed in a reaction solvent, in particular an aqueous solvent, especially water, or an organic solvent, especially as defined previously, in a proportion of from 0.25% to 25%, or even from 0.5% to 20%, or even from 1% to 15%, or even from 2% to 10%, or even from 3% to 8%, or even from 4% to 6%, and even more advantageously in a proportion of 5% by weight relative to the weight of the solvent.

A reaction solvent that is suitable for use in the invention for step c— may be chosen especially from an aqueous phase, especially water, or an organic phase, for example as defined above. Preferably, a reaction solvent that is suitable for use in the invention is an aqueous phase, and is more preferably water.

According to one embodiment, in a process of the invention, step c— may be performed at room temperature, and especially at a temperature ranging from about 20 to 24° C. Step c— may be performed, for example, for a time period of about 2 to 6 days, or even from 3 to 5 days, and preferably about 4 days.

Step c— may preferably be performed with stirring, according to the means usually used by a person skilled in the art in the field.

According to one embodiment, a process of the invention may also comprise the placing in contact of the nanoparticles obtained in step c— with at least one water-soluble polymer, under conditions favorable for immobilizing, especially by adsorption, this polymer at the surface of the functionalized nanoparticles.

According to a preferred embodiment variant, step c— may be performed in the presence of at least one water-soluble polymer, under conditions favorable for immobilizing, especially by adsorption, this polymer at the surface of the functionalized nanoparticles.

A water-soluble polymer that is suitable for use in the invention may especially be as defined previously.

A water-soluble polymer may be used in a solvent in a proportion of from 2% to 40% by weight relative to the weight of the solvent, or even from 3% to 30%, or even from 4% to 25%, or even from 5% to 20%, or even from 5% to 15%, or even from 6% to 12%, or even from 8% to 10%, and even more advantageously in a proportion of 10% by weight relative to the weight of the solvent.

A solvent that is suitable for use in the invention for the water-soluble polymer may especially be as defined above, and may preferably be water.

According to one embodiment, a process of the invention may also comprise an intermediate step between steps b— and c—, which consists in isolating the nanoparticles obtained on conclusion of step b—, especially by centrifugation, and optionally a step that consists in drying said isolated nanoparticles.

Such a step of isolating the nanoparticles may be performed by centrifugation at room temperature, and at a speed of between 4000 and 12 000 rpm, and in particular at about 8000 rpm (i.e. 8000±500 rpm), for a time of between 1 minute and 1 hour, especially between 2 minutes and 30 minutes and in particular for 10 minutes.

After the isolation step, the nanoparticles thus obtained may be subjected to a drying step, and then to a grinding step. The drying and grinding steps may be performed via any technique usually used by a person skilled in the art in the field.

According to one embodiment, a process of the invention may also comprise at least one additional step, on conclusion of step c—, which consists in crystallizing the nanoparticles. Such a step may be performed by dipping the dispersion of nanoparticles obtained in step c— into liquid nitrogen (N₂), dropwise. The precipitate thus obtained may be filtered off via any technique usually used by a person skilled in the art in the field.

According to one embodiment, a process of the invention may also comprise at least one additional step that consists in lyophilizing the nanoparticles. The lyophilization step may be performed via any technique usually used by a person skilled in the art in the field.

The nanoparticles thus obtained may be in the form of agglomerates that are self-dispersible in an aqueous or organic solvent, especially as defined previously.

A process of the invention advantageously makes it possible to obtain nanoparticles that are coated or modified at the surface with a silylated polymer, preferably as a monomolecular layer, and surface-functionalized with redox mediator molecules, these molecules being immobilized at the surface by means of non-covalent bonds, preferably interactions of π-π type, established between ethylenic, acetylenic and/or aromatic units respectively present on said silylated polymer and on said redox mediator molecule(s).

The present invention also relates to nanoparticles that may be obtained according to a process as defined above.

### Agglomerate

The nanoparticles described previously, and preferably the nanoparticles comprising at the surface at least one water-soluble polymer, may be formulated in the form of agglomerates.

Such an agglomerate may be in the form of beads, preferably having a mean diameter of about 2 to 3 mm.

According to one embodiment, an agglomerate of the invention may be self-dispersible in a hydrophilic solvent, preferably in an aqueous solvent, and more preferably in water.

The dispersion of an agglomerate of nanoparticles of the invention may advantageously be performed in an aqueous phase, and preferably in water, during the formulation of an ink.

The homogeneous nature of a solution or dispersion of an agglomerate of nanoparticles of the invention in a solvent may be evaluated via any method known in the field, for example, at the macroscopic scale, visually.

### Process for Preparing an Agglomerate

An agglomerate of the invention may be prepared more particularly according to a process as detailed below.

A process for preparing an agglomerate of the invention may comprise at least the steps consisting in:
providing a dispersion of nanoparticles of the invention, especially as defined previously, and preferably comprising at the surface at least one water-soluble polymer, especially as defined previously,
performing the dropwise dipping of said dispersion, in liquid nitrogen, to obtain a solid material,
recovering by filtration the solid material thus obtained, and, where appropriate,
lyophilizing the solid material thus recovered.

### Support Material

### Material

According to a preferred embodiment, the nanoparticles of the invention may be adsorbed onto the surface of a support material.

A support material that is suitable for use in the invention may be chosen from a multiwall carbon nanotube, a single-wall carbon nanotube, graphene, graphite, carbon fibers, and fullerenes.

Preferably, a support material that is suitable for use in the invention may be a multiwall carbon nanotube.

Carbon nanotubes that may be suitable for use in the invention may have a diameter of about 9.5 nm and a purity at least greater than or equal to 95%.

Multiwall carbon nanotubes that are suitable for use in the invention comprise at least two walls.

Preferably, a support material that is suitable for use in the invention is constituted by multiwall carbon nanotubes. Such nanotubes advantageously act as electron conductors, thus promoting the transfer of electrons from the catalytic site of an enzyme to an electrode, during the use of the nanoparticles of the invention in a fuel biocell.

According to one embodiment, at least one enzyme may also be adsorbed onto the surface of this support material. According to this embodiment, a support material of the invention may comprise, co-adsorbed onto the surface, at least nanoparticles of the invention and at least one enzyme.

### Enzyme

An enzyme that is suitable for use in the invention may be an enzyme that is capable of catalyzing an electron-generating reaction, and preferably an oxidation reaction.

Preferably, an enzyme that is suitable for use in the invention may be capable of catalyzing the oxidation of glucose. Such an enzyme may be chosen from glucose oxidase, cellobiose dehydrogenase and glucose dehydrogenase.

More preferably, an enzyme which is suitable for use in the invention may be glucose oxidase (GOx).

An enzyme that is suitable for use in the invention may be obtained via any method known in the field, such as extraction from microorganisms, and especially from bacteria or yeasts such as Aspergillus niger, producing them naturally or after modification by genetic engineering. Such methods are known to those skilled in the art and do not need to be presented in further detail herein.

### Process for Preparing a Support Material

A support material of the invention may be prepared more particularly according to the process as detailed below.

A process for preparing a support material of the invention may comprise at least the steps consisting in:
providing a dispersion of a support material in a physiologically acceptable solvent, preferably in an aqueous solution, and even more preferentially water,
providing a dispersion of nanoparticles functionalized with one or more molecules of a redox mediator as defined previously, and preferably comprising at the surface at least one water-soluble polymer, especially as defined previously, in a physiologically acceptable solvent, preferably an aqueous solution and even more preferentially water,
mixing the dispersion of the support material with the dispersion of nanoparticles under conditions favorable for the adsorption of the nanoparticles onto the surface of the support material.

For the purposes of the invention, the term “physiologically acceptable” is intended to denote a solvent that can be administered, for example, orally, topically or by injection, to an animal, preferably to a mammal and more preferably to a human being.

A support material that is suitable for use in the invention may especially be as defined previously.

Advantageously, the mixture of the dispersion of the support material with the dispersion of nanoparticles may be subjected to various mechanical, thermal or sonic agitation means. Preferably, an agitation means that is suitable for use in the invention may be sonic agitation, especially by ultrasonication. Such agitation advantageously makes it possible to promote the adsorption of the nanoparticles onto the surface of the support material, such as the walls of the carbon nanotubes. The duration of an ultrasonication bath may be, for example, about 30 minutes.

A process for preparing a support material of the invention may also comprise at least one additional step that consists in adding to the mixture of the dispersion of the support material with the dispersion of nanoparticles a solution or a dispersion of at least one enzyme, preferably as defined previously. The enzyme solution or dispersion may be prepared in a physiologically acceptable solvent, preferably an aqueous solution and even more preferentially water.

The mixture containing at least one support material, at least nanoparticles of the invention, and at least one enzyme may also be subjected to an agitation step as defined above. Such agitation advantageously makes it possible to promote the adsorption of the nanoparticles and of the enzyme onto the surface of the support material, such as the walls of carbon nanotubes. The duration of an ultrasonication bath may be, for example, about 30 minutes.

According to a preferred embodiment variant, the mixture of the support material, the nanoparticles of the invention and at least one enzyme may be prepared during the same step. The mixture thus obtained may also be subjected to an agitation step as defined above.

According to an embodiment variant, a process for preparing a support material of the invention may also comprise at least one step that consists in adding to the dispersion or suspension obtained at least one copolymer of fluorinated sulfonic acid. This step is advantageously performed prior to the agitation step.

According to one embodiment, the suspension or dispersion of a support material thus obtained may be used as ink.

### Ink

According to one embodiment, the nanoparticles of the invention may be formulated in the form of an ink.

An ink of the invention may comprise at least nanoparticles of the invention or at least one support material according to the invention.

The dispersing phase or solvent of an ink of the invention may be a hydrophilic solvent, preferably an aqueous or organic liquid phase, and more preferably water.

Preferably, a hydrophilic solvent that is suitable for use in the invention is a physiologically acceptable solvent.

According to one embodiment, an ink according to the invention may also comprise at least one fluoropolymer based on sulfonated tetrafluoroethylene copolymer.

A fluoropolymer based on sulfonated tetrafluoroethylene copolymer that is suitable for use in the invention may especially be Nafion®.

### Process for Preparing an Ink

An ink of the invention may more particularly be prepared according to a process as detailed below.

A process for preparing an ink of the invention may comprise at least the steps consisting in:
providing nanoparticles of the invention or a support material of the invention,
dispersing or suspending the nanoparticles or the support material in a hydrophilic solvent.

The ink thus obtained may be subjected to various mechanical, thermal or sonic agitation means. Preferably, an agitation means that is suitable for use in the invention may be sonic agitation, especially by ultrasonication. The duration of an ultrasonication bath may be, for example, about 30 minutes.

According to an embodiment variant, a process for preparing an ink of the invention may also comprise at least one step that consists in adding to the dispersion or suspension obtained at least one fluoropolymer based on sulfonated tetrafluoroethylene copolymer. This step is advantageously performed prior to the agitation step.

### Electrode

An electrode of the invention may be formed totally or partly from nanoparticles of the invention or from a support material of the invention, especially as defined previously.

According to one embodiment, the nanoparticles or the support material is/are deposited on a glass-like carbon support or an anionic and/or cationic membrane, preferably a membrane of a fluoropolymer based on sulfonated tetrafluoroethylene copolymer.

The choice of a material for an electrode of the invention may depend on several factors including, especially, the pH at which the enzyme-catalyzed reaction is to be performed or the nature of the redox mediator. A person skilled in the art may, on the basis of his general knowledge, make the appropriate choice of the material to be adopted.

For example, when the reaction catalyzed by the enzyme is to be performed, preferably, at a pH of 7 or close to 7, as is the case for GOx, a material that is suitable for use as an electrode of the invention may be a membrane of a fluoropolymer based on sulfonated tetrafluoroethylene copolymer, and preferably a Nafion® membrane.

Preferably, a support that is suitable for use as an electrode of the invention may be a membrane of a fluoropolymer based on sulfonated tetrafluoroethylene copolymer. A membrane of a fluorinated sulfonic acid copolymer that is suitable for use in the invention may be a Nafion® membrane.

### Process for Preparing an Electrode

An electrode of the invention may more particularly be prepared according to a process as detailed below.

A process for preparing an electrode of the invention may comprise at least one step that consists in depositing on a support, in particular a glass-like carbon support or an anionic and/or cationic membrane, preferably a membrane of a fluoropolymer based on sulfonated tetrafluoroethylene copolymer, an ink of the invention, especially as defined above.

The deposition of the ink may be performed via any technique known to those skilled in the art, and especially by drop-casting.

### Fuel Biocell

A biocell is formed from two electrodes. The anode transports the flow of electrons derived from the oxidation of glucose. These electrons are found at the end of the electrical circuit at the cathode and are taken up by a catalyst which performs the reduction of dioxygen to water. This flow of electrons, from the negative to the positive electrode, thereby induces the circulation of an electrical current in the external circuit.

A fuel biocell of the invention may comprise, as anode, or as anode compartment, nanoparticles of the invention, a support material of the invention, an ink of the invention, the ink being dried, or at least one electrode of the invention.

An ink of the invention may be deposited and dried on a support, for example a Nafion® membrane.

The anode compartment of a biocell of the invention may be fed with a continuous flow, by means of a peristaltic pump, of a glucose solution (for example 50 mM) prepared in a buffer, for example a phosphate buffer (100 mM, pH 7).

According to one embodiment, a biocell of the invention may comprise a cathode, or a cathode compartment, formed from an electrode comprising at the surface a metal chosen from platinum, gold and silver, especially in the form of metal nanoparticles. These nanoparticles may especially be present in a form immobilized on the surface of carbon nanoparticles.

Preferably, the metal may be platinum. Platinum advantageously makes it possible to maintain the biocompatibility of the biocell and also advantageously allows the reduction of atmospheric oxygen at a relatively high potential.

According to one embodiment, the metal may be deposited on the surface of a membrane of a fluoropolymer based on sulfonated tetrafluoroethylene copolymer.

According to one embodiment, the cathode of a biocell of the invention may also be covered with a porous metallic layer, and in particular with a porous layer of gold, silver, platinum or titanium.

The cathode compartment of a biocell of the invention may be obtained, for example, as follows. An ink based on carbon particles, for example Vulcan XC-72-R, covered with platinum nanoparticles, for example 60% Pt, may be used. The catalyst based on platinum nanoparticles may be sprayed onto a Nafion® membrane with a coverage of 600 μg/cm². A porous layer of gold deposited by PVD (physical vapor deposition) allows the collection of electrons.

The cathode compartment of the cell may be exposed to the air and may function under “air breathing” conditions.

Throughout the text, namely the description presented above and the examples presented below, the expression “between . . . and . . . ” relative to a range of values should be understood as including the limits of that range.

The examples given below are presented as illustrations of the subject of the invention and should not be interpreted as limiting its scope.

### EXAMPLES

### Example 1

### Synthesis of ZnO Nanoparticles Functionalized with Ferrocene

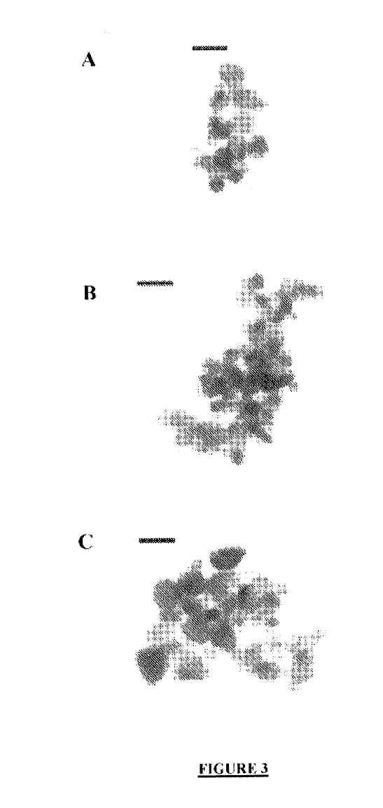

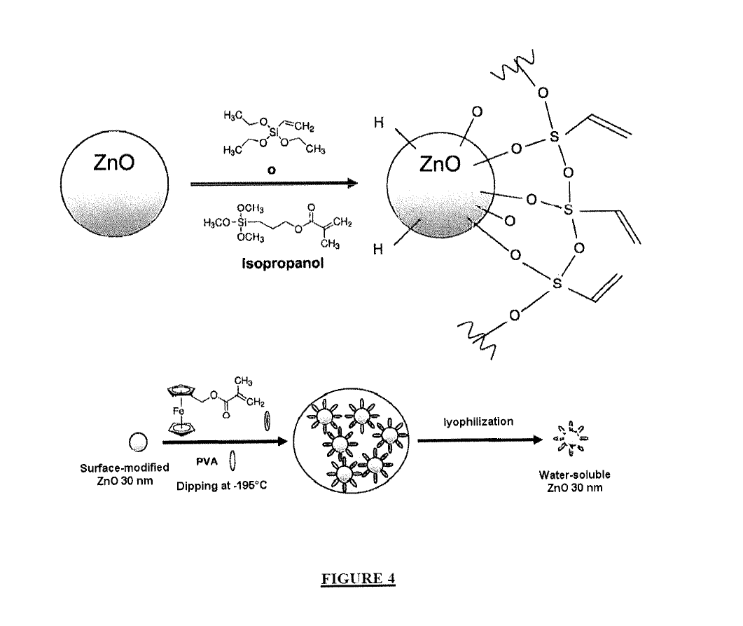

The scheme for synthesizing the ZnO nanoparticles functionalized with ferrocene according to the invention is presented in FIG. 3.

1a—Synthesis and Analysis of the ZnO Nanoparticles

The synthesis of pure ZnO nanoparticles was performed by coprecipitation.

The initial reagents are hydrated zinc nitrate and sodium carbonate. To prepare 10 g of a sample of pure ZnO, a solution containing 0.2 mol/L of Zn(NO₃)₉. 6H₂O is prepared, i.e. 36.5 g in 615 mL of distilled water. A solution containing 0.5 mol/L of sodium carbonate was prepared by dissolving 13.78 g of sodium carbonate in 246 mL of distilled water.

The zinc nitrate solution is added dropwise to the sodium carbonate solution with vigorous mechanical stirring at room temperature. The solution is then stirred for 2 hours at room temperature. The precipitate is filtered off through a Büchner funnel. The powder thus recovered is dried overnight in an oven at 100° C. The solid is then calcined in air (100 L/h) at 400° C. for 4 hours. The nanoparticles obtained are 20 to 30 nm in diameter, and are crystalline. Elemental analysis reveals the presence of zinc in the sample. The particle diameter measurements and the presence of Zn in the sample may be performed as described by Bellat et al. (Ind. Eng. Chem. Res., 2011, 50(9), 5714-5722).

1b—Surface Modification of the ZnO Nanoparticles

Nanometric ZnO (1 g) is dispersed in isopropanol (500 mL) in a 1 L round-bottomed flask with vigorous stirring and at room temperature. The hydrolyzing agent, aqueous ammonia (30%, 61 mL), is then added to the reaction mixture with vigorous stirring. A solution of triethoxyvinylsilane (0.323 g, 1.7 mmol, CAS No. 78-08-0) in isopropanol (250 mL) is added to the basic reaction mixture, and the resulting solution is stirred at room temperature for 24 hours. The solution is then centrifuged (8000 rpm, 10 min) and the supernatant is discarded. The solid obtained is dispersed in ethanol (100 mL) and then centrifuged again (8000 rpm, 10 min).

The functionalized ZnO is dried in an oven (50° C., 4 hours) and then ground and used as obtained for the following step.

1c—Preparation of ZnO Nanoparticles Functionalized with Ferrocene

A 100 mL aqueous solution containing polyvinyl alcohol PVA (CAS No. 9002-89-5) (10 g at 10 w % in deionized water) is stirred at room temperature. The surface-functionalized ZnO (0.5 g, 5 w %) is added with vigorous stirring, followed by ferrocenylmethyl methacrylate (0.5 g, 1.76 mmol, 5 w %, CAS No. 31566-61-7).

The reaction medium is stirred for 4 days at room temperature so as to obtain a homogeneous dispersion of the reaction medium. The dispersion obtained is dipped in liquid nitrogen (N₂) dropwise, filtered and then lyophilized for 48 hours. The resulting material (11 g, 100%) is in the form of beads of about 2-3 mm, which is self-dispersible in water.

### Example 2

### Preparation of a Biocell Based on ZnO Nanoparticles Functionalized with Ferrocene

2a—Principle

The cell is formed from two electrodes (FIG. 4). The anode transports the flow of electrons derived from the oxidation of glucose. These electrons are found at the end of the electrical circuit at the cathode and are taken up by a catalyst which performs the reduction of dioxygen to water. This flow of electrons, from the negative to the positive electrode, thereby induces the circulation of an electrical current in the external circuit.

In a cell according to the invention, the enzymatic catalyst for oxidizing glucose is glucose oxidase. The material used to make the cathode of the biocell is platinum, which has the advantage of maintaining the biocompatibility of the application. Platinum advantageously allows the reduction of atmospheric oxygen at a relatively high potential.

In general, the oxidation of glucose leads to the formation of gluconic acid according to the following reaction:

C₆H₁₂O₆→C₆H₁₀O₆+2H⁺+2e⁻

The reaction taking place at the cathode of the cell is the reduction of dioxygen to H₂O according to the following equation:

½O₂+2H⁺+2e⁻→H₂O

2b—Electrochemical Characterization of ZnO Nanoparticles Functionalized with Ferrocene (ZnO-Fc)

We began with the electrochemical characterization of ferrocene-functionalized ZnO nanoparticles alone. To do this, we prepare a solution with a concentration of 60 mg/mL of these nanoparticles in water. It should be noted that ferrocene is known to be insoluble in water.

The functionalization method of the invention allows perfect dispersion of the nanoparticles in water just after one hour in an ultrasonication bath.

10 μL of the ZnO solution prepared are deposited by “drop-casting” onto a glass-like carbon electrode (A35T090 analytical radiometer) 3 mm in diameter and then dried in air, and then under vacuum. This electrode constitutes our working electrode. Characterization by cyclic voltammetry of this compound is performed using a three-electrode system: a platinum counterelectrode, a saturated calomel electrode as reference electrode (XM110 analytical radiometer and REF621 analytical radiometer) and the functionalized glass-like carbon electrode as working electrode. The characterization is performed in a solution of phosphate buffer (100 mM) at pH 7. The sweep speed is 50 mV/s.

In order to improve the active and specific surface area, we used multiwall carbon nanotubes (MWCNT) (NANOCYL NC7000) supplied by Nanocyl. These carbon nanotubes are 9.5 nm in diameter and have a purity >95%. These carbon nanotubes are used without any additional purification step. For this, a dispersion in water formed from 60 mg/mL of ZnO-Fc and 2.5 mg/mL of MWCNTs was prepared. 10 μL of this dispersion were deposited on a glass-like carbon electrode and treated as mentioned previously.

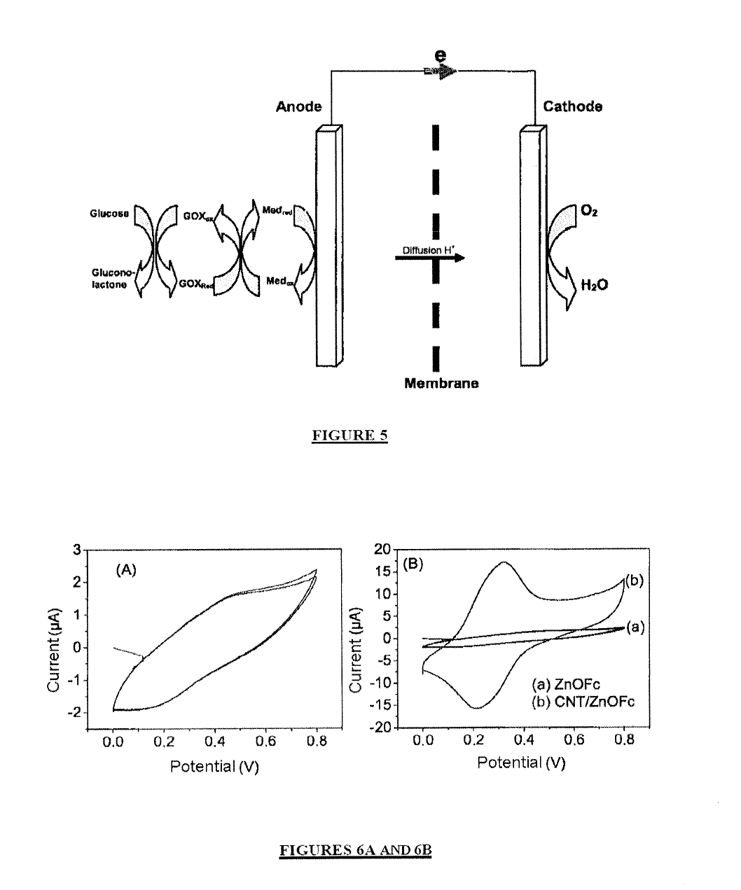

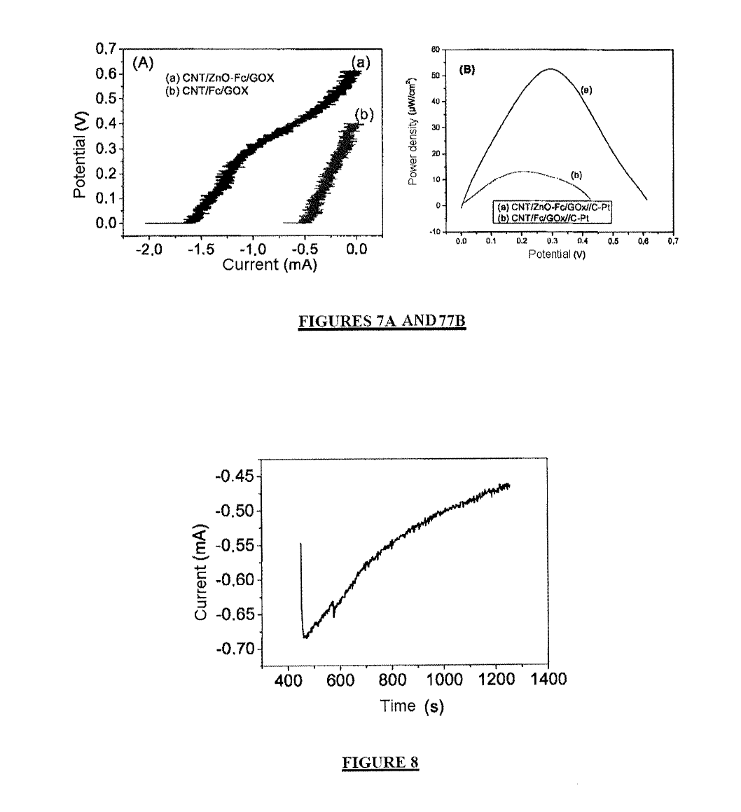

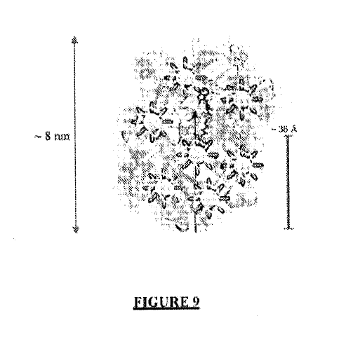

We observed the oxidation peak of ferrocene on the glass-like carbon electrode, thus confirming the presence of the redox mediator at the surface of the electrode (FIG. 5A). This anode oxidation peak increases considerably in the presence of carbon nanotubes (FIG. 5B).

This clearly shows the role played by the MWCNTs in terms of increasing the specific surface area and the electrical conductivity. Furthermore, we observed a shift in the oxidation peak toward positive potentials (Ea=300 mV) during the use of carbon nanotubes, which shows an improvement in the electron transfer promoted by the three-dimensional structure afforded by the CNTs.

2c—Design of Glucose Biocells Based on ZnO Nanoparticles Functionalized with Ferrocene (ZnO-Fc)

The ink based on ZnO nanoparticles functionalized with ferrocene is prepared as follows: a mixture of 60 mg/mL of ZnO-Fc and 2.5 mg/mL of MWCNT is treated in an ultrasonication bath for 1 hour. 7 mg/mL of glucose oxidase (GOX) from Aspergillus niger and 80 μL of Nafion® are added to this mixture. The suspension is once again treated for 30 minutes in an ultrasonication bath in order to homogenize the mixture and to allow adsorption of the nanoparticles and of the enzyme onto the walls of the carbon nanotubes.

To produce the cell, the ink based on ZnO-Fc catalyst is deposited on the anode part of a Nafion® membrane. For the cathode anode compartment of the cell, an ink based on carbon particles (Vulcan XC-72-R) covered with platinum nanoparticles (60% Pt) is used. The catalyst based on platinum nanoparticles is sprayed onto the Nafion® membrane with a coverage of 600 μg/cm².

A porous layer of gold deposited by PVD (physical vapor deposition) allows the collection of electrons.

The anode compartment is fed with a continuous flow by means of a peristaltic pump of a glucose solution (50 mM) prepared in the phosphate buffer (100 mM, pH 7). The cathode compartment of the cell is exposed to the air and functions under “air breathing” conditions.

The performance of the cell is tested under ambient conditions. To do this, positive potentials are applied between the anode and the cathode at a rate of 2 mV/s and a current was measured, indicating a circulation of electrons between the anode and the cathode as shown by the polarization curve. A comparative study of the performance of cells based on non-functionalized ferrocene was performed and clearly showed the role played by the ZnO nanoparticles in the catalytic process (FIGS. 6A and 6B).

Finally, discharge at a constant potential of 200 mV was undertaken for the cell based on CNTs/ZnO-Fc/GOX. At this potential, the current begins to fall to reach 460 μA after a period of 15 minutes (FIG. 7).

### BIBLIOGRAPHY

WO 2011/117357
WO 2005/096430
Bellat et al., Ind. Eng. Chem. Res., 2011, 50(9):5714-5722
Hu et al., J Mol Catal B-Enzym, 2011, 72:298
Liang et al., Electrochimica Acta, 2012, 69:167
Qiu et al., Biosensors & Bioelectronics, 2009, 24:2649
Qiu et al., Biosensors & Bioelectronics, 2009, 24:2920
Teng et al., Biosensors & Bioelectronics, 2011, 26:4661
Wilson et al., Biosensors & Bioelectronics, 1992, 7:165

1-19. (canceled)

20. Nanoparticles, at least partially surface-coated with a silylated polymer, and functionalized with one or more molecules of at least one redox mediator, in which the immobilization of said molecule(s) of said mediator at the surface of said nanoparticles takes place via non-covalent bonds, established between ethylenic, acetylenic and/or aromatic units, respectively, present on said silylated polymer and on said redox mediator molecule(s).

21. The nanoparticles as claimed in claim 20, said nanoparticles being nanoparticles of metal oxides.

22. The nanoparticles as claimed in claim 20, in which the immobilization of said molecule(s) of said mediator at the surface of said nanoparticles takes place via interactions of π-π type.

23. The nanoparticles as claimed in claim 20, in which the silylated polymer is obtained by polymerization of at least one precursor of said silylated polymer comprising at least one ethylenic, acetylenic and/or aromatic unit.

24. The nanoparticles as claimed in claim 23, in which said precursor is a silicon alkylalkoxide of general formula (I):
R¹R²R³SiR⁴
in which:
R¹, R² and R³, which may be identical or different, represent a group OR⁵ with R⁵ representing a saturated, linear or branched C₁ to C₄ alkyl group; or a halogen, and
R⁴ represents a linear, branched or cyclic C₂ to C₁₀ hydrocarbon-based group, comprising at least one ethylenic, acetylenic and/or aromatic unit, and, where appropriate, interrupted with one or more heteroatoms and/or bearing an oxo function.

25. The nanoparticles as claimed in claim 20, said nanoparticles being semiconductive.

26. The nanoparticles as claimed in claim 20, in which the redox mediator molecule(s) are chosen from ferrocenes; methylene green; Nile blue; macrocyclic organometallic complexes; quinones; phenoxazines; tetrathiafulvalene; tetracyanoquinodimethane; benzylviologen; tris(2,2′-bipyridine)cobalt(III) perchloride; indophenols.

27. The nanoparticles as claimed in claim 26, in which said mediator molecule(s) is/are present in a form functionalized with at least one radical comprising at least one ethylenic, acetylenic and/or aromatic unit.

28. The nanoparticles as claimed in claim 20, also comprising at the surface at least one water-soluble polymer.

29. The nanoparticles as claimed in claim 28, in which said water-soluble polymer is chosen from polyvinyl alcohol, polyethylene-40 stearate, poly(vinylidene chloride-co-vinyl chloride), poly(styrene-co-maleic anhydride), polyvinylpyrrolidone, poly(vinyl butyral-co-vinyl alcohol-co-vinyl acetate), poly(maleic anhydride-alt-1-octadecene), poly(vinyl chloride), PEOX (poly(2-ethyl-2-oxazoline)), PLGA (poly(lactic acid-co-glycolic acid)), and mixtures thereof.

30. An agglomerate of nanoparticles, said nanoparticles being at least partially surface-coated with a silylated polymer, and functionalized with one or more molecules of at least one redox mediator, in which the immobilization of said molecule(s) of said mediator at the surface of said nanoparticles takes place via non-covalent bonds, established between ethylenic, acetylenic and/or aromatic units, respectively, present on said silylated polymer and on said redox mediator molecule(s), said nanoparticles also comprising at the surface at least one water-soluble polymer.

31. A support material at the surface of which is adsorbed at least one nanoparticle, said nanoparticle being at least partially surface-coated with a silylated polymer, and functionalized with one or more molecules of at least one redox mediator, in which the immobilization of said molecule(s) of said mediator at the surface of said nanoparticle takes place via non-covalent bonds, established between ethylenic, acetylenic and/or aromatic units, respectively, present on said silylated polymer and on said redox mediator molecule(s).

32. The material as claimed in claim 31, at the surface of which is also adsorbed at least one enzyme, said enzyme(s) being capable of catalyzing an oxidation reaction.

33. An ink comprising at least nanoparticles or comprising a material at the surface of which is adsorbed at least one nanoparticle, said nanoparticles being at least partially surface-coated with a silylated polymer, and functionalized with one or more molecules of at least one redox mediator, wherein the immobilization of said molecule(s) of said mediator at the surface of said nanoparticles takes place via non-covalent bonds, established between ethylenic, acetylenic and/or aromatic units, respectively, present on said silylated polymer and on said redox mediator molecule(s).

34. An electrode totally or partially formed from nanoparticles or from a material at the surface of which is adsorbed at least one nanoparticle, said nanoparticles being at least partially surface-coated with a silylated polymer, and functionalized with one or more molecules of at least one redox mediator, wherein the immobilization of said molecule(s) of said mediator at the surface of said nanoparticles takes place via non-covalent bonds, established between ethylenic, acetylenic and/or aromatic units, respectively, present on said silylated polymer and on said redox mediator molecule(s).

35. A fuel biocell comprising, as anode, nanoparticles, a material at the surface of which is adsorbed at least one nanoparticle, an ink comprising at least nanoparticles or comprising a material at the surface of which is adsorbed at least one nanoparticle, said ink being dried, or at least one electrode totally or partially formed from nanoparticles or from a material at the surface of which is adsorbed at least one nanoparticle,
said nanoparticles being at least partially surface-coated with a silylated polymer, and functionalized with one or more molecules of at least one redox mediator, wherein the immobilization of said molecule(s) of said mediator at the surface of said nanoparticles takes place via non-covalent bonds, established between ethylenic, acetylenic and/or aromatic units, respectively, present on said silylated polymer and on said redox mediator molecule(s).

36. A process for preparing nanoparticles, said nanoparticles being at least partially surface-coated with a silylated polymer, and functionalized with one or more molecules of at least one redox mediator, wherein the immobilization of said molecule(s) of said mediator at the surface of said nanoparticles takes place via non-covalent bonds, established between ethylenic, acetylenic and/or aromatic units, respectively, present on said silylated polymer and on said redox mediator molecule(s), comprising at least the steps consisting in:
a—providing nanoparticles, dispersed in an organic solvent,
b—placing said nanoparticles in contact with at least one silylated polymer precursor comprising at least one ethylenic, acetylenic and/or aromatic unit, under conditions favorable for the formation of a silylated polymer bearing reactive ethylenic, acetylenic and/or aromatic unit(s), and for its deposition at least partially at the surface of said nanoparticles,
c—placing the nanoparticles obtained in step b— in contact with at least one redox mediator molecule bearing at least one ethylenic, acetylenic and/or aromatic unit, under conditions suitable for immobilizing said molecule(s) of said mediator at the surface of said nanoparticles via the establishment of non-covalent bonds, established between the ethylenic, acetylenic and/or aromatic units respectively present on said silylated polymer and said redox mediator molecule(s).

37. The process as claimed in claim 36, in which step b— is performed in the presence of a hydrolyzing agent, chosen especially from aqueous ammonia, sodium hydroxide and potassium hydroxide.

38. The process as claimed in claim 36, in which step c— is performed in the presence of at least one water-soluble polymer, under conditions favorable for immobilizing this polymer on the surface of said nanoparticles.

39. Nanoparticles that may be obtained according to a process comprising at least the steps consisting in:
a—providing nanoparticles, dispersed in an organic solvent,
b—placing said nanoparticles in contact with at least one silylated polymer precursor comprising at least one ethylenic, acetylenic and/or aromatic unit, under conditions favorable for the formation of a silylated polymer bearing reactive ethylenic, acetylenic and/or aromatic unit(s), and for its deposition at least partially at the surface of said nanoparticles,
c—placing the nanoparticles obtained in step b— in contact with at least one redox mediator molecule bearing at least one ethylenic, acetylenic and/or aromatic unit, under conditions suitable for immobilizing said molecule(s) of said mediator at the surface of said nanoparticles via the establishment of non-covalent bonds, established between the ethylenic, acetylenic and/or aromatic units respectively present on said silylated polymer and said redox mediator molecule(s).

40. A process for preparing an agglomerate of nanoparticles, comprising at least the steps consisting in:
providing a dispersion of nanoparticles, said nanoparticles being at least partially surface-coated with a silylated polymer, and functionalized with one or more molecules of at least one redox mediator, in which the immobilization of said molecule(s) of said mediator at the surface of said nanoparticles takes place via non-covalent bonds, established between ethylenic, acetylenic and/or aromatic units, respectively, present on said silylated polymer and on said redox mediator molecule(s), said nanoparticles also comprising at the surface at least one water-soluble polymer,
performing the dropwise dipping of said dispersion, in liquid nitrogen, to obtain a solid material,
recovering by filtration the solid material thus obtained, and, where appropriate
lyophilizing the solid material thus recovered.

41. A process for preparing an electrode, comprising at least one step that consists in depositing onto a support, an ink comprising at least nanoparticles or comprising a material at the surface of which is adsorbed at least one nanoparticle, said nanoparticles being at least partially surface-coated with a silylated polymer, and functionalized with one or more molecules of at least one redox mediator, wherein the immobilization of said molecule(s) of said mediator at the surface of said nanoparticles takes place via non-covalent bonds, established between ethylenic, acetylenic and/or aromatic units, respectively, present on said silylated polymer and on said redox mediator molecule(s).
